(19) United States
(12) Patent Application Publication
(10) Pub. No.: US 2007/0233090 A1
(43) Pub. Date: Oct. 4, 2007

(54) ALIGNING CROSS-CONNECTOR

(76) Inventors: Bill R. Naifeh, Dallas, TX (US); Carolyn Rogers, Frisco, TX (US)

Correspondence Address: CARR LLP (IST), 670 FOUNDERS SQUARE, 900 JACKSON STREET, DALLAS, TX, 75202, US

(21) Appl. No.: 11/678,465

(22) Filed: Feb. 23, 2007

Related U.S. Application Data
(60) Provisional application No. 60/775,879, filed on Feb. 23, 2006.
(60) Provisional application No. 60/814,943, filed on Jun. 19, 2006.
(60) Provisional application No. 60/775,877, filed on Feb. 23, 2006.
(60) Provisional application No. 60/814,753, filed on Jun. 19, 2006.
(60) Provisional application No. 60/814,753, filed on Jun. 19, 2006.
(60) Provisional application No. 60/826,763, filed on Sep. 25, 2006.
(60) Provisional application No. 60/863,284, filed on Oct. 27, 2006.

(51) Int. Cl. A61F 2/30

(52) U.S. Cl. 606/61

(57) ABSTRACT

There is disclosed at least one adjustable cross connector comprising two curved members which couple to each other in a slideable fashion, wherein the free ends are adapted to couple with a rod or another member of a spine stabilization system.

### CROSS-REFERENCED TO RELATED APPLICATIONS

The present application is related to and claims priority from the following commonly assigned patent applications: U.S. Provisional Patent Application 60/775,879, entitled “Aligning Cross-Connector,” filed on Feb. 23, 2006; and U.S. Provisional Patent Application 60/814,943, entitled “Aligning Cross-Connector,” filed on Jun. 19, 2006. The disclosures of which are hereby incorporated by reference.

The present application is related to the following commonly assigned patent applications: U.S. patent application Ser. No. 10/914,751, entitled “System and Method for Dynamic Skeletal Stabilization,” filed on Aug. 9, 2004; U.S. Provisional Patent Application 60775877, entitled “Multi-Level Spherical Linkage Implant System,” filed on Feb. 23, 2006; U.S. patent application Ser. No. 11/443,236, entitled “System and Method for Dynamic Skeletal Stabilization,” filed on May 30, 2006; U.S. Provisional Patent Application 60814753, entitled “Multi-Level Spherical Linkage Implant System,” filed on Jun. 19, 2006; U.S. patent application Ser. No. 11/467,798, entitled “Alignment Instrument for Dynamic Spinal Stabilization Systems,” filed on Aug. 28, 2006; U.S. Provisional Patent Application 60826763, entitled “Alignment Instrument for Dynamic Spinal Stabilization Systems,” filed on Sep. 25, 2006; U.S. Provisional Patent Application 60863284, entitled “Alignment Instrument for Dynamic Spinal Stabilization Systems,” filed on Oct. 27, 2006; and U.S. patent application Ser. No. ______, entitled “MULTI-LEVEL SPHERICAL LINKAGE IMPLANT SYSTEM” filed on Feb. 23, 2007; the disclosures of which are hereby incorporated by reference.

### FIELD OF THE INVENTION

This disclosure relates to skeletal stabilization and, more particularly, to systems and methods for dynamic stabilization of human spines.

### BACKGROUND

The human spine is a complex structure designed to achieve a myriad of tasks, many of them of a complex kinematic nature. The spinal vertebrae allow the spine to flex in three axes of movement relative to the portion of the spine in motion. These axes include the horizontal (bending either forward/anterior or aft/posterior), roll (bending to either left or right side) and vertical (twisting of the shoulders relative to the pelvis).

In flexing about the horizontal axis, into flexion (bending forward or anterior) and extension (bending backward or posterior), vertebrae of the spine rotate about the horizontal axis to various degrees of rotation. The sum of all such movement about the horizontal axis of produces the overall flexion or extension of the spine. For example, each of the vertebra that make up the lumbar region of the human spine move through roughly an arc of 15° relative to its adjacent or neighboring vertebrae. Vertebrae of other regions of the human spine (e.g., the thoracic and cervical regions) have different ranges of movement. Thus, if one were to view the posterior edge of a healthy vertebrae, one would observe that the edge moves through an arc of some degree (e.g., of about 15° in flexion and about 5° in extension if in the lumbar region) centered around an elliptical center of rotation. During such rotation, the anterior (front) edges of neighboring vertebrae move closer together, while the posterior edges move farther apart, compressing the anterior of the spine. Similarly, during extension, the posterior edges of neighboring vertebrae move closer together, while the anterior edges move farther apart, compressing the posterior of the spine. Also during flexion and extension, the vertebrae move in horizontal relationship to each other, providing up to 2-3 mm of translation.

In a normal spine, the vertebrae also permit right and left lateral bending. Accordingly, right lateral bending indicates the ability of the spine to bend over to the right by compressing the right portions of the spine and reducing the spacing between the right edges of associated vertebrae. Similarly, left lateral bending indicates the ability of the spine to bend over to the left by compressing the left portions of the spine and reducing the spacing between the left edges of associated vertebrae. The side of the spine opposite the compressed portion is expanded, increasing the spacing between the edges of vertebrae comprising that portion of the spine. For example, the vertebrae that make up the lumbar region of the human spine rotate about an axis of roll, moving through roughly an arc of 10° relative to its neighbor vertebrae, throughout right and left lateral bending.

Rotational movement about a vertical axis relative to the portion of the spine moving is also desirable. For example, rotational movement can be described as the clockwise or counter-clockwise twisting rotation of the vertebrae during a golf swing.

The inter-vertebral spacing (between neighboring vertebrae) in a healthy spine is maintained by a compressible and somewhat elastic disc. The disc serves to allow the spine to move about the various axes of rotation and through the various arcs and movements for normal mobility. The elasticity of the disc maintains spacing between the vertebrae, allowing room or clearance for compression of neighboring vertebrae, during flexion and lateral bending of the spine. In addition, the disc allows relative rotation about the vertical axis of neighboring vertebrae, allowing twisting of the shoulders relative to the hips and pelvis. Clearance between neighboring vertebrae maintained by a healthy disc is also important to allow nerves from the spinal chord to extend out of the spine, between neighboring vertebrae, without being squeezed or impinged by the vertebrae.

In situations (based upon injury or otherwise) where a disc is not functioning properly, the inter-vertebral disc tends to compress, and in doing so pressure is exerted on nerves extending from the spinal cord by this reduced inter-vertebral spacing. Various other types of nerve problems may be experienced in the spine, such as exiting nerve root compression in the neural foramen, passing nerve root compression, and ennervated annulus (where nerves grow into a cracked/compromised annulus, causing pain every time the disc/annulus is compressed), as examples. Many medical procedures have been devised to alleviate such nerve compression and the pain that results from nerve pressure. Many of these procedures revolve around attempts to prevent the vertebrae from moving too close to each other thereby maintaining space for the nerves to exit without being impinged upon by movements of the spine.

In one such procedure, screws are embedded in adjacent vertebrae pedicles and rigid rods or plates are then secured between the screws. In addition other devices, such as a fusion cage or spacer, may be inserted in-between the adjacent vertebrae to aid in fusing the vertebrae together. In such a situation, the pedicle screws (which are in effect extensions of the vertebrae) and rods serve to distract the degenerated disc space, maintaining adequate separation between the neighboring vertebrae, so as to prevent the vertebrae from compressing the nerves. This prevents nerve pressure due to extension of the spine; however, when the patient then tries to bend forward (putting the spine in flexion), the posterior portions of at least two vertebrae are effectively held together. Furthermore, the lateral bending or rotational movement between the affected vertebrae is significantly reduced, due to the rigid connection of the spacers. Overall movement of the spine is reduced as more vertebras are distracted by such rigid spacers. This type of spacer not only limits the patient's movements, but also places additional stress on other portions of the spine (typically, the stress placed on adjacent vertebrae without spacers being the worse), often leading to further complications at a later date.

In other procedures, dynamic fixation devices may be used to preserve some motion of the spine while still distracting the vertebrae to relieve pressure placed on the various nerves. However, dynamic fixation devices may require additional stability. Furthermore, some systems might require alignment during implantation.

What is needed is additional stability for use in dynamic or fusion systems while increasing the ease of insertion by allowing for alignment and adjustability of components during implantation.

### SUMMARY

In response to these and other problems, there is presented certain aspects which may provide methods and systems for providing additional stability to spine stabilization. For instance, there is disclosed at least one adjustable cross connector comprising two curved members which couple to each other in a slideable fashion, wherein the free ends of the adjustable cross connector are adapted to couple with a rod or another member of a spine stabilization system.

These and other features, and advantages, will be more clearly understood from the following detailed description taken in conjunction with the accompanying drawings. It is important to note the drawings are not intended to represent the only aspect of the invention.

### BRIEF DESCRIPTION OF THE DRAWINGS

For a more complete understanding of the present invention and the advantages thereof, reference is now made to the following Detailed Description taken in conjunction with the accompanying drawings, in which:

FIG. 1 is a perspective view of one possible embodiment of a system incorporating a cross-connector and a pair of dynamic stabilization systems;

FIG. 2A is an enlarged perspective view of one possible embodiment of a cross-connector which may be used in conjunction with a dynamic stabilization system;

FIG. 2B is a perspective view of one possible embodiment of a first elongated member of the cross-connector shown in FIG. 2A.

FIG. 2C is top view of the cross-connector illustrated in FIG. 1;

FIG. 3 is a perspective view of one possible embodiment of an alignment device attached to a cross-connector and dynamic stabilization system;

FIG. 4A is a perspective view of one possible embodiment of an adapter which may be used with a cross-connector and an alignment rod;

FIG. 4B is a perspective view of an alternative embodiment of an adapter which may be used with a cross-connector and an alignment rod;

FIG. 5A is a an exploded view of an alternative embodiment of a cross-connector;

FIG. 5B is a top view of the cross-connector illustrated in FIG. 5A.

### DETAILED DESCRIPTION

For the purposes of promoting an understanding of the principles of the present inventions, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the inventions as described herein are contemplated as would normally occur to one skilled in the art to which the invention relates.

In some embodiments, a cross-connector is disclosed that may be utilized for both aligning and providing additional stability to one or more dynamic stabilization constructs. A dynamic stabilization construct may be placed on each side of the spinous process of the spine. The cross connector may then secure the two or more dynamic stabilization constructs together to provide additional stabilization. The cross-connector may be designed to attach to a dynamic stabilization construct(s) and thereafter be adjusted as to align with the spine's natural center of rotation or other location as desired by a surgeon. Once the cross connector is aligned, it may be secured together and may remain in place as part of the stabilization system. The cross connector and dynamic stabilization construct together may provide additional stability to the spine and may aid in permitting a substantial range of motion in flexion, extension, rotation, anterior-posterior translation and/or other desired types of spinal motion. The cross connector device disclosed below may be used with any dynamic of fusion system.

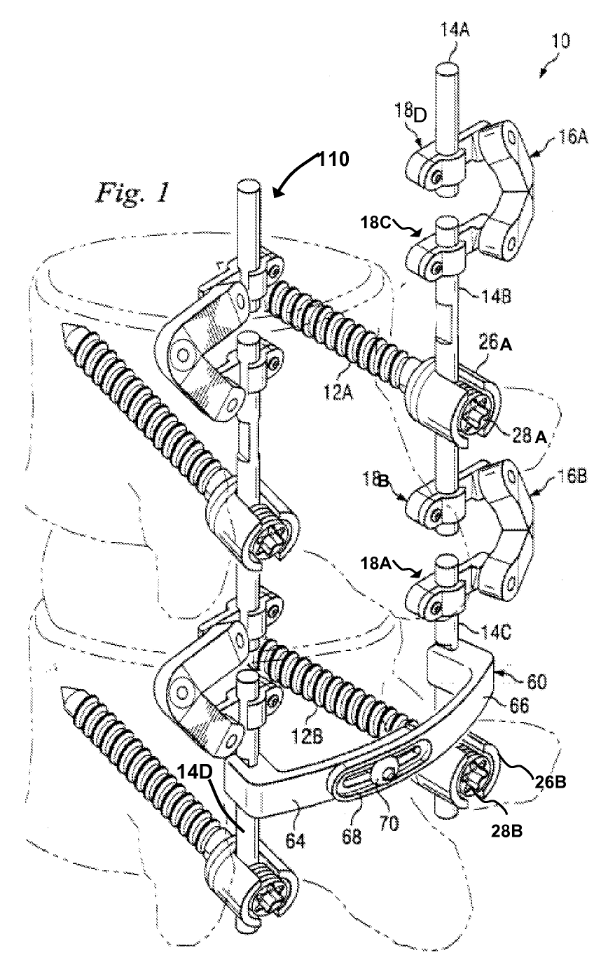

Referring to FIG. 1, one embodiment of a system is shown that may incorporate a cross connector 60 secured between two spine stabilization constructs 10 and 110. Similar spine stabilization constructs are disclosed in further detail pending patent application Ser. No. ______, entitled “MULTI-LEVEL SPHERICAL LINKAGE IMPLANT SYSTEM” filed on Feb. 23, 2007 and in pending U.S. patent application Ser. No. 60/775,877 entitled “MULTI-LEVEL SPHERICAL LINKAGE IMPLANT SYSTEM,” filed Feb. 23, 2006, the contents of which are incorporated herein by reference.

For purposes of illustration, only the spine stabilization construct 10 will be described in detail. The spine stabilization construct 110 contain similar components and will not be described in detail. Furthermore, for purposes of clarity, only a portion of the spine stabilization constructs 10 and 110 are illustrated in FIG. 1.

In certain embodiments, the spine stabilization construct 10 may incorporate a plurality of bone anchors 12A, 12B and 12C (bone anchor 12C is not shown for purposes of clarity). The bone anchors 12A-12C each have a distal end which secures to a patient's vertebrae. In certain embodiments, the proximal end of the bone anchors 12A-12C may secure directly or indirectly to one or more rods 14A-14C in a polyaxial manner (the connection between bone anchor 12C and rod 14A is not shown in FIG. 1 for purposes of clarity). As illustrated, the rods 14B and 14C may couple to cylindrical heads 26A-26B which may be multi-axially coupled to the bone anchors 12A-12B (respectively). In certain embodiments, the cylindrical heads 26A-26B may have an aperture that is dimensioned to receive one or more rods 14B-14C. The rods 14B-14C may be able to slide within the aperture of cylindrical heads 26A-26B and move along multiple axis relative to the bone anchors 12A-12B to allow for proper alignment and easy installation.

In certain embodiments, one or more dynamic braces 16A-16B may be located between two or more bone anchors 12A-12B. The dynamic braces 16A-16B may be coupled to respective rods 14A-14C which may couple to bone anchors 12A-12C. In certain embodiments, the dynamic braces 16A-16B may be offset from a longitudinal axis extending between two adjacent bone anchors. The offset may provide additional spacing for the dynamic braces 16A-16B so that the dynamic braces 16A-16B do not interfere with the neighboring anatomy of the spine. The offset of the dynamic braces 16A-16B may be positioned towards either side of the longitudinal axis of two adjacent bone anchors.

Dynamic braces 16A-16B may be coupled directly to respective rods 14A-14C or connecting members 18A-18D may be used to couple the rods 14A-14C to dynamic braces 16A-16B (respectively). The connecting members 18A-18D may enable the braces 16A-16B to be adjusted axially along the rods 14A-14C. The connecting members 18A-18D may also allow for rotational movement with respect to the rods 14A-14C. The connecting members 18A-18D thus may allow for increased adjustability of dynamic braces 16A-16B and rods 14A-14C. This adjustability may allow the surgeon to align and place various components of dynamic stabilization construct 10 (and 110) more easily. Once the desired axial position and angulation of the braces 16A-B are achieved the rods 14A-14C and the cylindrical heads 26A-26B may be fastened securely to bone anchors 12A-12B by a locking elements 28A-28B. Locking elements 28A-28B may be threaded locking caps or collets, or other suitable locking elements known to those skilled in the art. After dynamic stabilization construct 10 is implanted on one side of the spinous process, the procedure detailed above may be repeated on the opposing side of the spinous process for dynamic stabilization construct 110.

In certain embodiments, after two or more opposing dynamic stabilization constructs 10 and 110 are secured to the spine, a cross-connector 60 may be used to further stabilize the opposing dynamic stabilization constructs 10 and 110. The cross connector 60 may attach to the constructs 10 and 110 such that the cross connector 60 does not interfere the motion of dynamic braces 16A-16B. For example, FIG. 1 shows the cross connector 60 attached to two opposing rods 14C and 14D. The dynamic braces 16A and 16B may thus be partially stabilized by cross connector 60, while not hindering the natural controlled motion of dynamic braces 16A and 16B.

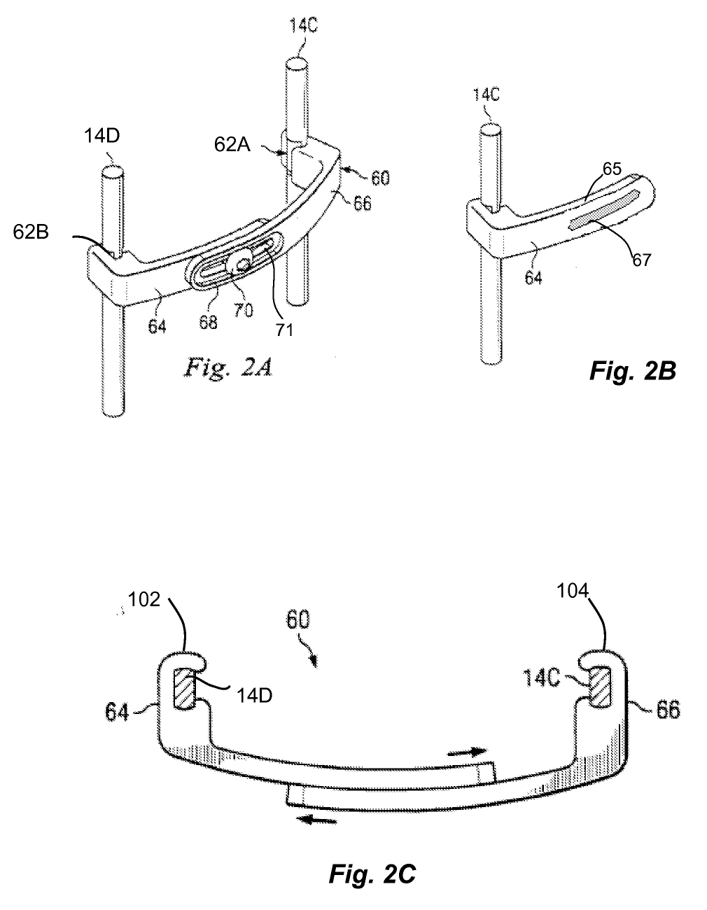

Referring now to FIGS. 2A-2C, one embodiment of the cross-connector 60 of FIG. 1 is illustrated in greater detail. The opposing rods 14C and 14D may have gripping features 62A and 62B which may aid in securing the rods 14C and 14D to cross connector 60, as shown in FIG. 2A. These gripping features 62A and 62B may include not only indentations as shown in FIG. 2A, but may also include a section of the rod having a different cross sectional geometries, such as rectangular, hexagonal, octagonal or hemi circular. Protrusions and indentations of various shapes and geometries may be located on the rod to aid in the attachment of the cross-connector 60 to the rods 14C and 14D. The rods 14C and 14D may also have a rough surface texture to aid in rigidly securing the cross connector 60 to the rods 14C and 14D.

In certain embodiments, the cross-connector 60 may incorporate two or more elongated members 64 and 66. The first and second elongated members 64 and 66 may have a rod gripping portion 102 and 104 at their exterior ends. In certain embodiments, the rod gripping portion 102 and 104 of the first and second elongated members 64 and 66 may secure to the rods 14C and 14D (respectively) by a snap-fitting around the gripping features 62A and 62B. As illustrated in FIG. 2C, the rod gripping portions 102 and 104 may have a hook shape which may interface with gripping features 62A and 62B to aid in capturing rod 14C and 14D. In certain embodiments, the rods 14C and 14D may be further secured to elongated members 64 and 66 by inserting a threaded fastener (not shown), such as a set screw, through elongated members 64 and 66 such that the set screw presses against rods 14C and 14D respectively.

FIG. 2B is a detailed view of one embodiment of the first elongated member 64. In some embodiments, the first elongated member 64 may have a curved elongated portion 65.

In certain embodiments an elongated recess or groove 67 may extend partially into the top surface of the first elongated member. The elongated recess or groove 67 may extend longitudinally along the first elongated member 64 to allow for an almost infinite number of adjustable positions for the first and second elongated members. Similarly, the second elongated member 66 may have also have an elongated curved portion and an elongated recess 68 (FIG. 2A) that extends into the top surface of the second elongated member 66. The elongated recess 68 may extend longitudinally along the second elongated member 66 to allow for adjustability of the longitudinal position of the first and second elongated members 64 and 66. In certain embodiments, the elongated recess 68 may receive a fastener 70 to lock the first and second elongated members 64 and 66 together. The fastener 70 may have a distal threaded section and a proximal head section. The elongated recess 68 may be dimensioned so that the proximal head section of fastener 70 is flush or below the top surface of second elongated member 66 to prevent fastener 70 from interfering with neighboring anatomy of the patient's spine. An elongated slot 71 may be located within recess 68 which extends through the bottom surface of second elongated member 66. The threaded section of fastener 70 may pass through elongated slot and into groove 67 on the first elongated member 64. In other embodiments threaded section of fastener 70 may lock onto the top surface of first elongated member 64.

In certain embodiments the elongated portion of the first elongated member 64 may be temporarily pivotably and slidingly mated to the elongated portion of the second elongated member 66. The adjustability of first and second elongated members 64 and 66 may allow cross connector 60 to accommodate the spine anatomy of patients of all sizes and bone structures. The first member 64 and second member 66 may telescope or slide across each other (as shown in FIG. 2C) enabling adjustment of the rods 14C and 14D for alignment of the spine stabilization system 10 and 110. The radius of curvature of the top section of the first member 64 may be substantially the same as the radius of curvature of the bottom section of the second elongated member 66 which may aid in the smooth controlled pivoting and sliding of the two elongated members 64 and 66 relative to each other. Once the desired angle and longitudinal position of the two elongated members 64 and 66 are achieved the fastener 70 may be inserted into the recess 68 and such that fastener 70 extends through the elongated slot 68 on the second elongated member and contacts the top surface or the groove 67 of the first elongated member to lock the two elongated members 64 and 66 together.

In certain embodiments, cross connector 60 may be preassembled with the bottom surface of the second elongated member 66 mated to the top section of the first elongated member 64. The fastener 70 may be partially inserted into the recess 68 and hand tightened such that a small compressive force acts on the top surface (or the groove 67) of the first elongated member 64 so that first and second elongated members 64 and 66 may still slide and pivot relative to each other. Once the desired position of cross connector 60 is achieved during implantation, the fastener 70 may be tightened with an instrument to rigidly and permanently secure the first and second elongated members 64 and 66 together.

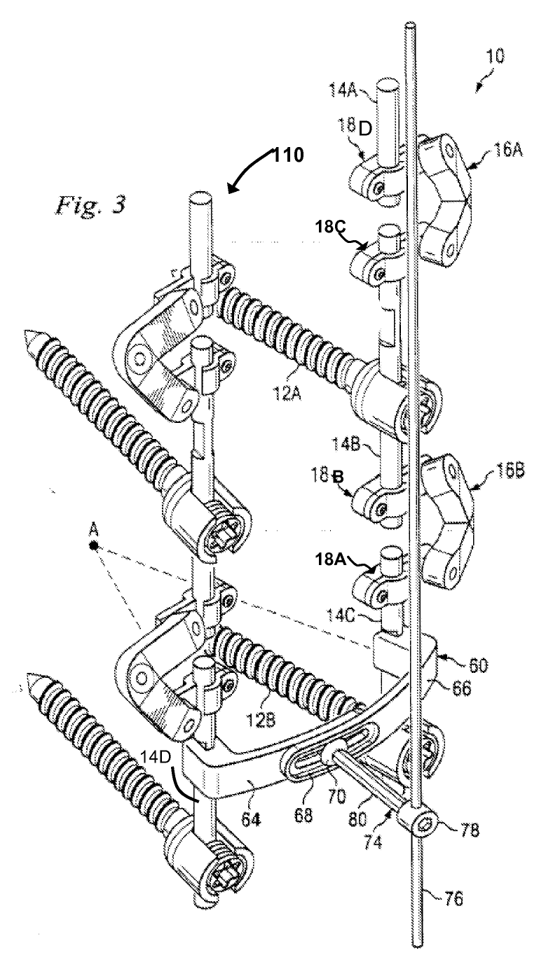

Referring now to FIG. 3, one possible embodiment of the cross-connector 60 is illustrated connected to the spine stabilization constructs 10 and 110 and aligned with a center of rotation, which is illustrated as point A. As explained in detail in U.S. patent application Ser. Mo. 11/467,798, entitled “Alignment Instrument for Dynamic Spinal Stabilization Systems,” filed on Aug. 28, 2006; U.S. Provisional Patent Application 60826763, entitled “Alignment Instrument for Dynamic Spinal Stabilization Systems,” filed on Sep. 25, 2006; and U.S. Provisional Patent Application 60863284, entitled “Alignment Instrument for Dynamic Spinal Stabilization Systems,” filed on Oct. 27, 2006, the dynamic links 16A and 16B may be aligned to rotate about a center of rotation. Thus, in certain embodiments the cross-connector 60 may also be sized to align to a center of rotation.

As illustrated in FIG. 3, an adapter 74 may be coupled to the fastener 70. In certain embodiments, adapter 74 may have a distal portion for engaging the fastener 70 or the cross connector 60 and a proximal portion for engaging an alignment device, such as alignment rod 76. In other embodiments, the proximal portion of the adapter 74 may include a head 78 for the attachment of other devices and components. The alignment rod 76 may be inserted into a head 78 of the adapter 74 and thereafter used to align the cross connector 60 with point A. In certain embodiments, the alignment rod 76 may be connected to other adapters 74 and other cross connectors (not shown) along the spine to provide proper alignment. Once the cross connectors and/or the stabilization system 10 and 110 are aligned with point A, the adapter may be used to tighten the fastener 70. Once fastener 70 is tightened, the elongated members 62 and 64 may be secured from moving in respect to each other, thereby setting the stabilization system 10 and 110 into substantially permanent alignment with point A.

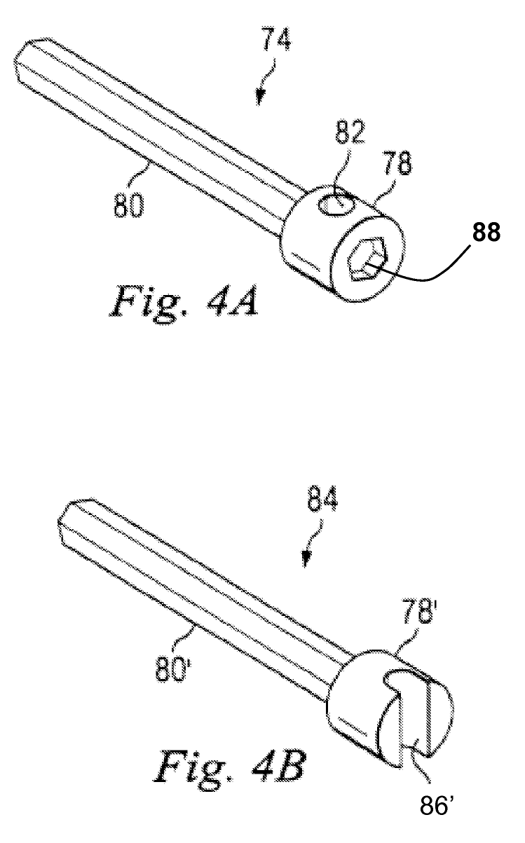

Referring to FIG. 4A, there is illustrated one embodiment of the adapter 74 shown in FIG. 3 that may be used in conjunction with the cross-connector. In this embodiment, the adapter 74 may have an elongated body 80 enabling the adapter 74 to extend away from any anatomy that may interfere with aligning the cross connector 60. The elongated body 80 may couple to the head 78. The head 78 may include an orifice 82 that extends in a direction generally transverse to the longitudinal axis of adapter 74. The orifice 82 may be dimensioned to slidingly receive the alignment rod 76 as shown in FIG. 3. A torque transfer feature 88 may be located on head 78 and may interface with a torque transfer device, such as a driver, to aid in positioning adapter 74 and/or tightening fastener 70 to cross connector 60.

Referring to FIG. 4B, an alternative embodiment of an adapter 84 is illustrated. Many of the portions of the adapter 84 may be substantially similar in construction and function to the portions of the adapter 74. Such similar component parts are designated in FIG. 4B with the same reference numerals utilized above in the description of the adapter 74, but are differentiated therefrom by means of a prime (′) designation. The adapter 84 may differ from the adapter 74 in that, for example, the adapter 84 comprises a different head 78′. An elongated body 80′ may have a proximal portion that may attach to the head 78′. The distal portion of elongated body may couple to cross connector 60 to aid in alignment of the cross-connector 60 and dynamic stabilization constructs 10 and 110. The head 78′ of the adapter 84 may have a channel 86′ extending into the top surface of head 78′ which may receive the alignment rod 76 such that alignment rod 76 is positioned generally transverse with respect to the longitudinal axis of the adapter 84. The channel 86′ may also interface with a torque transfer device to aid in the insertion of fastener 70.

In certain embodiments the adapter 74 (or 84) and the alignment rod 76 may be manufactured from metallic materials such as stainless steel, nitinol or titanium. Polymers may also be used to manufacture adapters 74 and 84 and alignment rod 76. The specific material may be chosen based the surgeon's desire for the device appear on a fluoroscopy image during surgery which may aid the surgeon in aligning the dynamic stabilization devices 10 and 110 and cross connector 60.

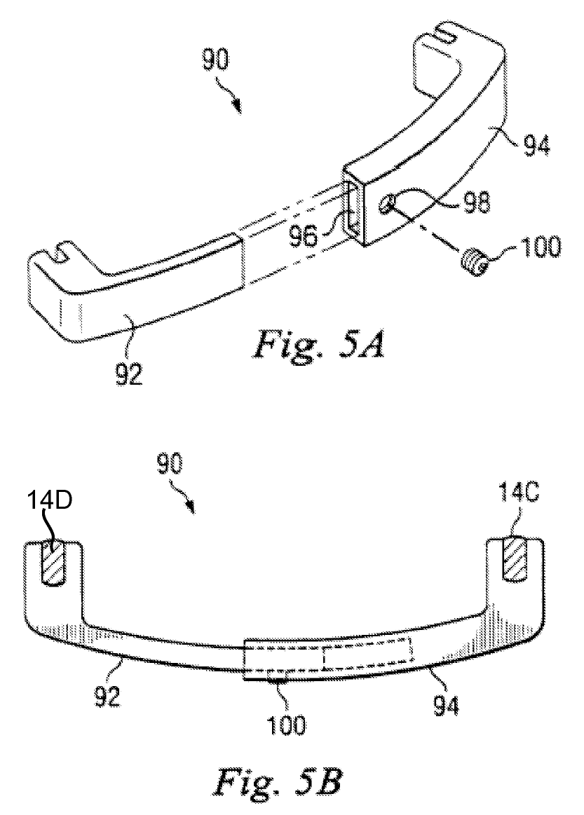

Referring to FIGS. 5A and 5B, another embodiment of a cross-connector 90 is illustrated. The cross-connector 90 may be substantially similar in function but may differ (e.g., in construction) from the cross-connector illustrated in FIGS. 1 through 3 as described above. For example, the cross-connector 90 may differ from the cross-connector 60 in that cross-connector 90 may comprise a first elongated member 92 which fits within a second elongated member 94. In certain embodiments, the channel 96 may be substantially straight to receive first elongated member 92 and allow proper sliding between the two members.

In other embodiments the channel 96 may have curved top and bottom surfaces that correspond to curved surfaces on the first elongated member 92. The channel 96 may be sufficiently oversized as to allow a gap between the first elongated member 92 and the second elongated member 94. The gap may allow the two elongated members to slide freely and pivot a controlled degree (based on the size of the gap). The gap may be increased or decrease depending on the desired amount of motion between the first and second elongated members.

As described above, cross connector 90 may be similar to cross connector 60. For example, the first and second elongated members 92 and 94 may have a section with a convex top surface and a concave bottom surface to accommodate neighboring anatomy of the spine.

In certain embodiments the cross connector 90 may have first and second elongated members with exterior gripping portions to attach to the rods 14C and 14D as described in earlier embodiments. The gripping portions may have a hook geometry that snap fits around the rods 14C and 14D to maintain the position of cross connector 90 while the surgeon secures cross connector 90 into its final position. A set screw may also be used to further secure cross connector 90 to rods 14C and 14D as described in the other embodiments above. In other embodiments cross-connector 90 may secure onto the rods 14C and 14D of the spine stabilization system 10 and 110 by fasteners which lock the first and second elongated members to the rods without the need for a snap fit type interlock.

In certain embodiments the second elongated member 96 may have an orifice 98 for receiving a fastener 100 for securing the first and second elongated member 92 and 94 of cross connector 90 together once the desired position of cross-connector 90 is achieved. Once fastener 100 is secured in place cross connector 90 may stabilize and support the spine stabilization constructs 10 and 110 in proper alignment while allowing natural motion of the spine. The cross connector 90 may be aligned in the same manner as cross connector 60 as described above. The fastener 100 may engage adapters 74 and 84 to aid in the use of the alignment rod 76.

The foregoing description of the embodiments of the invention has been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

What is claimed is:

1. A system for dynamic stabilization of the spine comprising:
at least two pairs of opposing bone anchors, each bone anchor comprising a distal vertebral anchoring portion and a proximal cylindrical head having two longitudinal slots together defining two branches, the cylindrical head having an internal screw thread;
a first and second opposing dynamic braces, each dynamic brace having a proximal section and a distal static section coupled within the cylindrical head and a dynamic portion positioned between the proximal and the distal static sections;
at least one cross connector comprising, a first member having an elongated portion and a distal gripping portion coupled to the first dynamic brace, the elongated portion having a concave top section having a first radius of curvature along a portion of the longitudinal axis of the first member, a convex bottom section having a first radius of curvature along a portion of the longitudinal axis of the first member, an elongated aperture extending into the top section and of the first member,
a second member having a distal gripping portion coupled the second dynamic brace and an elongated portion temporarily pivotably and slidingly mated to the elongated portion of the first member, the elongated portion having a concave top section with a first radius of curvature along a portion of the longitudinal axis of the second member, a convex bottom section having a first radius of curvature along a portion of the longitudinal axis of the second member, an elongated recess extending partially through the top section of the second member and an elongated aperture located within the recess that extends through the bottom section of the second member,
a locking member positioned within the elongated recess and extending through both the elongated aperture of the first member and the elongated aperture of the second member.
